(19) United States
(12) Patent Application Publication
(10) Pub. No.: US 2023/0324381 A1
(43) Pub. Date: Oct. 12, 2023

(54) Label-free, real-time, whole-cell response monitoring with liquid Raman spectroscopy

(71) Applicant: The Board of Trustees of the Leland Stanford Junior University, Stanford, CA (US)

(72) Inventors: Loza Fekadu Tadesse, Los Altos Hills, CA (US); Jack HU, Stanford, CA (US); Amr Ahmed Essawi Saleh, Palo Alto, CA (US); Stefanie S. Jeffrey, Los Altos Hills, CA (US); Jennifer A Dionne, Menlo Park, CA (US)

(21) Appl. No.: 18/022,201

(22) PCT Filed: Sep. 1, 2021
(86) PCT No.: PCT/US2021/048668

§ 371 Date: Feb. 20, 2023

Related U.S. Application Data
(60) Provisional application No. 63/073,320, filed on Sep. 1, 2020.

(51) Int. Cl. G01N 33/569; G01N 15/14; G01N 33/543

(52) U.S. Cl. CPC G01N 33/56911; G01N 2015/0065; G01N 33/54346; G01N 15/1434

(57) ABSTRACT

Improved surface enhanced Raman spectroscopy (SERS) of biological targets in liquids is provided. Nanoparticles are treated with a surfactant to provide an electrostatic attraction between the nanoparticles and the biological targets. The resulting clustering of the nanoparticles at the biological targets improves the SERS signal, Such SERS spectroscopy enables real time monitoring of the biological targets, thereby enabling a wide variety of assays etc.

### FIELD OF THE INVENTION

This invention relates to Raman spectroscopy of biological samples.

### BACKGROUND

Real-time whole-cell Raman spectroscopy is a useful tool to monitor cellular responses to multiple perturbing stimuli. These stimuli may include chemicals, drugs, biomolecules, cell-cell interactions, and microenvironmental conditions (such as oxygen, pH, mechanical factors, extracellular vesicles). One application of such spectroscopy is rapid and real-time determination of a patient's susceptibility to drugs, for disease diagnosis and treatment. Drug susceptibility testing is generally a lengthy process, spanning hours to days. It also usually requires hundreds to millions of cells, which are obtained through culturing or propagation. This process is not only naturally slow and sometimes challenging, but can select for certain subpopulations of heterogeneous cells. Existing drug susceptibility platforms that work at the single-cell level generally are destructive or require labels, such that the same cell cannot be interrogated multiple times with distinct drugs, drug combinations, or varying drug concentrations. Accordingly, it would be an advance in the art to provide improved Raman spectroscopy of biological samples.

### SUMMARY

A rapid, drug susceptibility testing platform at the few-to-single-cell level is especially important in metastatic cancer treatment, infectious disease diagnosis, neurodegenerative disorders, diabetes, and others. Below, two exemplary applications are highlighted, to provide context.

### Example 1

### Critical Problem in Determining Drug Susceptibility of Metastatic Cancer

Metastatic cancer presents an intractable clinical problem. Metastases often involve multiple sites throughout the body, with cells that are spatially and temporally heterogeneous. Metastases tend to be molecularly discordant from the original primary tumor and from each other, yet the first line treatment of metastatic disease is often based on biomarkers present within the primary tumor that may have been excised years earlier or from a needle biopsy of a single metastasis. When tumor cells become resistant to such first line treatments, as occurs in most patients, then second, third, and even fourth line treatments are empirically selected until metastatic tumor cells no longer respond to any known therapies. Unfortunately, predictive drug biomarkers that are clinically proven to forecast cancer cell response are either nonexistent for the majority of chemotherapeutics or inadequate for many targeted biologic agents and immunotherapeutics. Treatment of metastatic cancer could be revolutionized if doctors and clinicians could screen metastases for drug sensitivity throughout disease progression, for example using circulating tumor cells (CTCs) or needle biopsies. These cells represent a snapshot of cancer cells that persist after previous drug treatments, and reflect in real-time cancer cells from multiple metastatic sources with diverse genomic, epigenomic, transcriptional, and metabolomic molecular characteristics. However, only single to double digit numbers of CTCs are typically present in a single blood draw, challenging drug susceptibility testing.

### Example 2

### Critical Problem in Treating Infectious Diseases

In the realm of infectious diseases, accurate and rapid antibiotic and antiviral susceptibility testing is crucial to ensure proper use of drugs. The WHO predicts that antibiotic resistance will claim 10 million lives per year and will be the most prevalent cause of death by 2050. Current methods for infectious disease drug susceptibility testing generally include phenotypic approaches such as disk diffusion methods, broth microdilution methods, antimicrobial gradient strips, and automated AST tools, such as the Vitek 2. These conventional assays generally take about 12 hours. Additionally, they can only be performed on a limited number of drugs for a given sample. Recently, it has been shown that combinations of antibiotics (for example, mixtures of two or more antibiotics) can be more potent than using individual drugs, enabling the use of lower doses and the treatment of otherwise drug resistant bacteria. Such permutations cannot be conducted with conventional assays, given the large number of possible permutations of antibiotics for a particular patient infection. Another key limitation of conventional assays is that they give binary results (that is, susceptible; or not susceptible; for a given antibiotic concentration) and cannot, in a single-step, determine the MIC for a particular pathogen; instead, the tests must be run multiple times for varying antibiotic concentrations. Finally, these assays do not elucidate the particular molecular-level interaction between pathogens and antibiotics. Revealing such interactions in real-time could help elucidate the molecular mechanism by which pathogens, including patient-specific pathogens, respond to antibiotics and develop resistance.

This work addresses these major limitations of existing drug susceptibility platforms, enabling single-cell drug testing in real time, with the opportunity for sequential drug testing on the same single cancer cell. Embodiments of the invention also span beyond drug testing to real-time cellular monitoring of other environmental perturbations.

One embodiment is a platform enabling real-time monitoring of hundreds down to single cells originating from broad biological fluids including less viscous fluids like blood plasma to more viscous fluids, such as sputum. A liquid chamber was created for live-cell Surface-enhanced Raman spectroscopy (SERS) measurements, and utilizes nanoparticles to enhance the SERS signature to achieve signal at the few-to-single-cell level. Surface-enhanced Raman spectroscopy utilizes Raman scattering—an inelastic photon scattering off molecules. This Raman signature can be used to monitor changes in the cellular behavior under different circumstances. Addition of plasmonic substrates enhances the naturally weak Raman scattering phenomena (1 in 10⁶ photons). A liquid chamber platform was built for Raman-based monitoring of cells and cell responses in different controlled environments. The liquid cell sample is pipetted to the well, then sealed with a cover slip. The liquid chamber can include a support glass, Si, or metallic substrate, for SERS interrogation. The hole size can be varied to accommodate desired volume of sample. Additional modification can be made, such as introducing inflow and outflow tubes, to allow for dynamic fluid introduction. For plasmonic-based liquid SERS, gold nanorods were synthesized following standard protocols in large volumes at desired aspect ratios matching spectral regions of both the incident laser light and the Raman scattered light for efficient Raman signal enhancement in liquid. The nanorods and cells are mixed prior to placement in the liquid chamber. This approach has the advantage of compatibility with existing microfluidic platforms, such as Fluidigms platforms.

In one embodiment, the liquid SERS platform is able to analyze cells in physiologically relevant environments, such as in plasma, serum, or even whole-blood. In another embodiment, methods can be applied to drug susceptibility testing at the few to single-cell level both for research and clinical diagnostic laboratories. From a patient sample (such as a blood draw or sputum sample), the assay would determine the optimal and selective treatment at any given point, not only at the start of the disease, but also as the disease progresses. Commercial applications include, but are not limited to:
Drug and antibiotic susceptibility tests on mammalian and bacterial cells, respectively. The platform is designed to work at the multiple or single-cell level, it is especially useful for cells that are difficult to culture, such as in cancer, microbiome studies, neurodegenerative disease studies, and environmental microbiology.
Drug development, drug repurposing, and drug use in clinical trials.
Monitoring single-cell drug response in the presence of other environmental stimuli such as pH, oxygen concentration, and temperature variations.
Studying cell-cell communication and interactions (such as immune cell-tumor cell interactions) in the presence of various drugs or post-vaccination.

Embodiments of the invention can be advantageous in some or all of the following manners:
Rapid, real-time interrogation of cells.
Non-destructive.
The cells remain live and intact.
Label-free.
No additional tags (dyes, probes, etc.) are required.
Operation at the few-to-single-cell level.
Ability to test multiple drugs and environmental influences on the same cell

Most in vitro drug screening assays are performed on thousands to tens-of-thousands of cells. The use of multiplex fluorescent-based probes or single-cell mass cytometry—based on time of flight mass spectroscopy imaging with targeted probes—represent exciting developments for probing cell biology and cell phenotyping with use of up to 50 tagged probes, but are performed on fixed and permeabilized or frozen cells, so cannot be applied to real-time drug testing of the same single cell. Moreover, propagation of CTCs in culture or in mouse models is difficult, may produce varying results depending on culture condition, and is frequently unsuccessful (1-33% success rate) and propagation conditions may select for subpopulations of heterogeneous CTCs, which could impact drug testing results.

The platform of this work enables liquid biopsy-based single-cell assay to predict and directly measure the response of a patient's cancer cells to therapy. The assay can be performed on live cells without the need for fixing or freezing the cells and additional propagation steps at few-to-single-cell level. This assay can be applied at many time points throughout the course of disease. In contrast to biopsying metastases in internal organs, blood samples can be readily obtained multiple times before, during, and after systemic therapy enabling continuous monitoring. Even a single positive CTC provides independent prognostic information for late clinical recurrence following primary treatment. Moreover, CTCs reflect the diversity of residual tumor cells from multiple concurrent metastatic sites and may show differences from biomarkers present in the original tumor. The platform allows real-time drug testing on CTCs throughout the course of the disease, especially following evidence of disease progression, enabling optimal and selective treatment. By helping select more appropriate treatments at any given time, the assay could help turn metastatic cancer into a long-term chronic disease rather than a disease associated with a short-term, poor outcome.

In the realm of antibiotic susceptibility testing, current methods include phenotypic approaches monitoring cellular changes to co-incubation with antibiotics such as diffusion, dilution, and automated tools and genotype-based approaches, including polymerase chain reaction (PCR) and

DNA microarray. These approaches have significant drawbacks including long incubation times, large reagent volumes, tedious experimental steps, operator induced errors, and large capital investment. The platform allows for direct fingerprinting of few to single cells to determine their species and antibiotic susceptibility profile directly from their Raman spectra. Furthermore, combination therapy is gaining traction as a more effective treatment approach compared to single antibiotic treatments. The platform can be used to test the effect of combined antibiotic therapy on pathogens by monitoring dynamic changes in the bacteria directly from Raman spectral changes.

### BRIEF DESCRIPTION OF THE DRAWINGS

FIGS. 1A-B schematically show electrostatic attraction between nanoparticles and biological targets.

FIG. 1C schematically shows the experimental arrangement of the work of section B.

FIGS. 1D-E show characterization of the nanorods of the work of section B.

FIGS. 2A-C show SERS results of the work of section B.

FIGS. 3A-F show dependence of SERS results on nanorod aspect ratio.

FIGS. 4A-F are images showing the dependence of nanorod clustering at cell surfaces on cell type.

FIG. 5 shows zeta function results for the nanorods of the work of section B.

FIG. 6 is a table of nanorod concentrations of the work of section B.

### DETAILED DESCRIPTION

Section A describes general principles relating to embodiments of the invention. Section B describes a detailed example relating to characterization of live bacteria in liquid media. Section C is supplemental information for the example of section B.

### A) General Principles

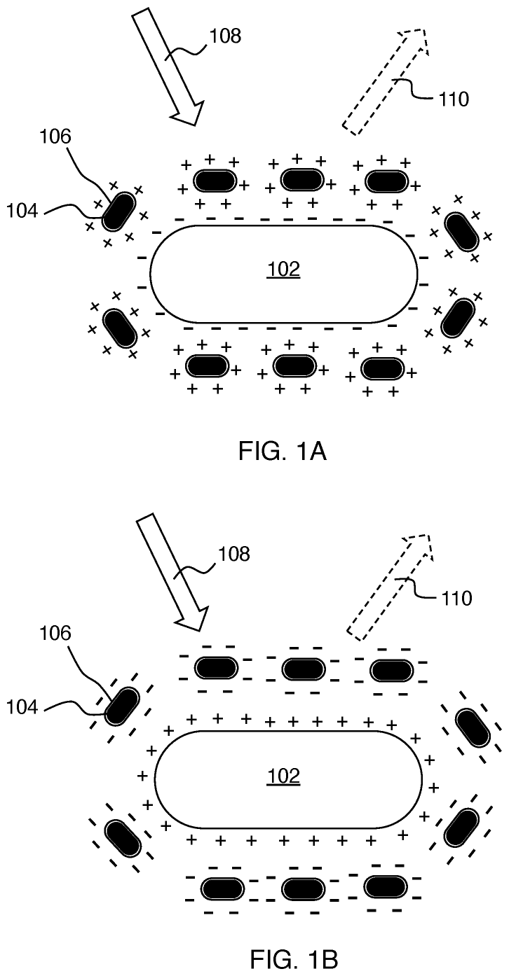

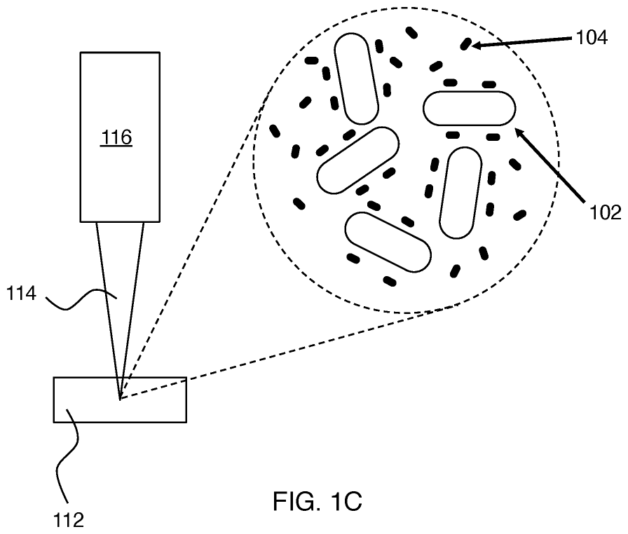

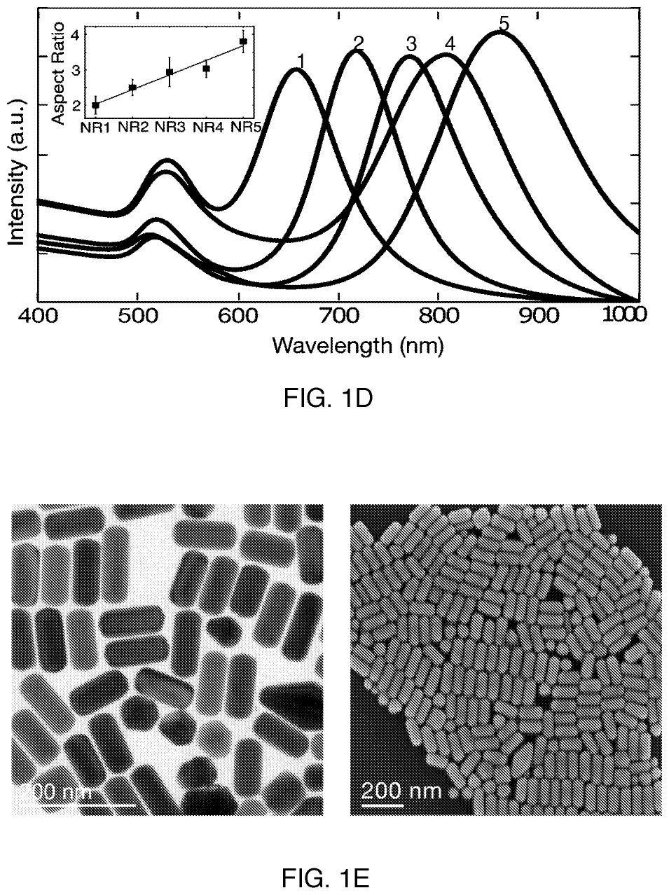

FIG. 1A schematically shows operation of a first embodiment of the invention. This example is a method of monitoring one or more biological target species in a liquid. Here 102 is a cell or the like of the target biological species. The target biological species can be any cellular or non-cellular biological species, including but not limited to: mammalian cells, cancer cells, bacteria, viruses, nucleic acids and proteins. In all the listed biological species surface charge is present and will be employed for targeted selective enhancement. For nanoscale biological species such as viruses, nucleic acids and proteins, it is expected that the biological species will be surrounding a nanoparticle.

Nanoparticles 104 are dispersed in the liquid. The nanoparticles 104 are treated with a surfactant 106 configured to provide an electrostatic attraction between the nanoparticles and at least a selected species of the one or more biological target species. Here the selected species are one or more species in the sample that are of interest for being monitored, assayed etc. Although surfactant 106 is shown on FIG. 1A as a distinct layer coating nanoparticles 104, it is also possible for this surfactant to be provided by a surface treatment of the nanoparticles that provides electrostatic attraction as above, without necessarily forming an identifiable separate coating layer on the nanoparticles. This electrostatic interaction is shown on FIG. 1A as negative surface charge on biological target 102 and positive surface charge on nanoparticles 104. FIG. 1B shows the alternative of positive surface charge on biological target 102 and negative surface charge on nanoparticles 104. Thus, in cases where the selected species has a negative surface charge, the surfactant is configured to provide a positive surface charge. Similarly, in cases where the selected species has a positive surface charge, the surfactant is configured to provide a negative surface charge. The electrostatic attraction between the nanoparticles and the selected species is preferably label-free. Nanoparticles 104 can be metallic such as gold and silver, dielectric such as silicon and titanium oxide or a mixture of metallic and dielectric material such as silicon coated with gold.

The liquid is illuminated with Raman pump light 108 and Raman-scattered light 110 from the liquid is observed. The Raman-scattered light 110 is scattered by the selected species in an interaction that is surface-enhanced by the nanoparticles.

Practice of the invention does not depend critically on the liquid in which the biological target species is found. Suitable liquids include, but are not limited to: plasma, serum, whole blood, saliva and sputum. Practice of the invention also does not depend critically on details of how the fluid is handled. One example is given below in section B, but the general principle is that any handling of the liquid should not interfere with the intended monitoring of the selected species.

The observing Raman-scattered light from the liquid can be performed continuously in real time for a viewing duration.

The Raman scattered light can be from a single living cell of the selected species.

The selected species can be a bacterium species. Then

Raman spectroscopy as described herein can be used to perform an assay of one or more antibiotics vs. the bacterium species using the Raman-scattered light.

The selected species can be a cancer cell species. Then Raman spectroscopy as described herein can be used to perform an assay of one or more anti-cancer agents vs. the cancer cell species using the Raman-scattered light.

The selected species can be a mammalian cell species. Then Raman spectroscopy as described herein can be used to perform an assay of one or more drugs vs. the mammalian cell species using the Raman-scattered light.

### B) Detailed Example

Rapid bacterial detection, identification, and antibiotic susceptibility testing is a critical clinical challenge. Standard bacterial diagnostics requires culturing steps that are naturally slow, accompanied by separate identification and antibiotic susceptibility tests that together span several days. Molecular diagnostic tools such as polymerase chain reaction (PCR), matrix-assisted laser desorption/ionization time-of-flight mass spectrometry (MALDI-TOF), and lateral flow enzyme immunoassays (EIA) can expedite the process, but still require hundreds of thousands of cells, significant capital investment, and/or cell-specific labels for high-fidelity testing; they are also destructive tests, and hence are ill-suited for real-time drug susceptibility testing.

Vibrational spectroscopy-based approaches such as Raman and Infrared spectroscopy promise culture-free, label-free, fast, accurate, and sensitive identification of bacteria with minimal sample preparation and without significant cell damage. These techniques provide a spectroscopic fingerprint of bacterial cells, with signal enhancements afforded by plasmonic substrates via either surface-enhanced Raman spectroscopy (SERS) or Infrared Absorption Spectroscopy (SEIRA). For portable, low-cost diagnostics, SERS has gained particular traction: the visible-frequency excitation and detection of SERS allows single-cell-level sensitivity as well as cheaper detectors and light sources. Further, its integration with microfluidics along with the development of paper-based SERS assays and portable Raman microscopes increases its accessibility for clinical application. Still, while considerable advances in the field of bacteria identification with SERS have been shown, many studies to date use dried samples due to the ease of sample preparation and spectral acquisition. In contrast, clinical samples are generally in liquid state as drying can be detrimental to the cells, removes or modifies important biological information from the SERS spectra, and does not allow dynamic study on the effect of antibiotics on bacterial cells.

Although clinically desirable, liquid Raman measurements present several challenges. Scattering from the inhomogeneous bacterial liquid with variations in dielectric constants leads to signal loss. Liquid SERS also requires devising easy-to-use and safe (i.e. free of hazardous bacterial exposure) platforms with optically compatible components. Efforts have been made to enable SERS measurements from liquid droplets on substrates. However, the droplets were open to air, increasing the risk of aerosol/droplet exposure to the individuals performing measurements on pathogenic bacteria. Additionally, droplets also tend to dry over a period of minutes, possibly providing insufficient interrogation time for bacteria identification and especially antibiotic susceptibility testing. This approach also limits the number of cells that can be analyzed to the size of the droplet. To advance liquid SERS, microfluidic approaches have gained traction; here, the flow rate even at the single-cell level can be synchronized with the Raman signal acquisition for achieving targeted cell sorting as demonstrated in yeast strains Saccharomyces cerevisiae. This approach, however, can only handle small sample volumes. Additionally, the strong Raman background from the commonly used PDMS material interferes with weak Raman signals from smaller cell types like bacteria compared to yeast. Moreover, for all existing liquid SERS approaches, there remains the need to understand and optimize plasmonic particle and bacterial cell interaction for efficient, uniform SERS enhancement.

Here we demonstrate a liquid bacterial SERS platform with consistent, large-area SERS enhancement. In particular, we investigate the effects of bacterial surface charge, gold-nanorod aspect ratio, and nanoparticle concentration to systematically determine the optimal parameters for liquid SERS. We use Gram-negative E. coli and S. marcescens, and Gram-positive S. aureus and S. epidermidis as model bacteria to study their interaction with gold nanorods. By optimizing the nanorod to bacteria concentration ratios, we obtain SERS signatures from liquid bacteria samples with signal intensities uniformly enhanced to detectable levels (compared to no signal with bacteria in water) across a 100×200 μm² area. Interestingly, the enhancement does not significantly vary with distinct nanorod aspect ratios, regardless of overlap with the laser excitation and Raman region of the bacteria; however, Gram-positive bacteria do show more significant enhancement. Cryo-electron microscopy (cryo-EM) reveals variations in nanoparticle binding affinities to Gram-positive and—negative bacterial types, depending on the charge of the bacteria membrane. As such, the bacteria with higher surface charge density also exhibit significantly higher SERS signal. Our work lays a foundation for performing, optimizing, and understanding liquid SERS measurements from biological fluids, en-route to clinical SERS and real-time drug susceptibility testing.

FIGS. 1C-E are an overview of the liquid-SERS chamber and plasmonic nanoparticles employed. FIG. 1C shows liquid well imaging setup with bacteria 102 and nanorods 104 both suspended in water and mixed together in the well 112. Here 116 schematically shows the objective lens of a confocal Raman microscope, and 114 is the 785 nm incident light for the Raman spectroscopy. FIG. 1D shows absorption spectra of nanorods in the liquid well with longitudinal plasmon resonances ranging from 670-860 nm. The inset shows aspect ratios plotted against peak resonance wavelength. The region from ˜835-915 nm represents the bacterial Raman shift region. FIG. 1E shows TEM (left) and SEM (right) images of synthesized gold nanorod particles used for liquid SERS measurement, showing monodispersity in nanorod size and shape.

We created a liquid chamber (hereafter “well”) for live bacterial SERS measurements as schematically shown by 112 in FIG. 1C. The well in our experiments included a silicon wafer base, a ˜100 μm tall spacer layer, and a glass coverslip; double-sided adhesive was used for the spacer layer, punched using single paper hole punchers to create reproducible liquid well sizes. The liquid bacterial sample is pipetted into the well, then sealed with a cover slip and placed on a microscope stage for SERS imaging.

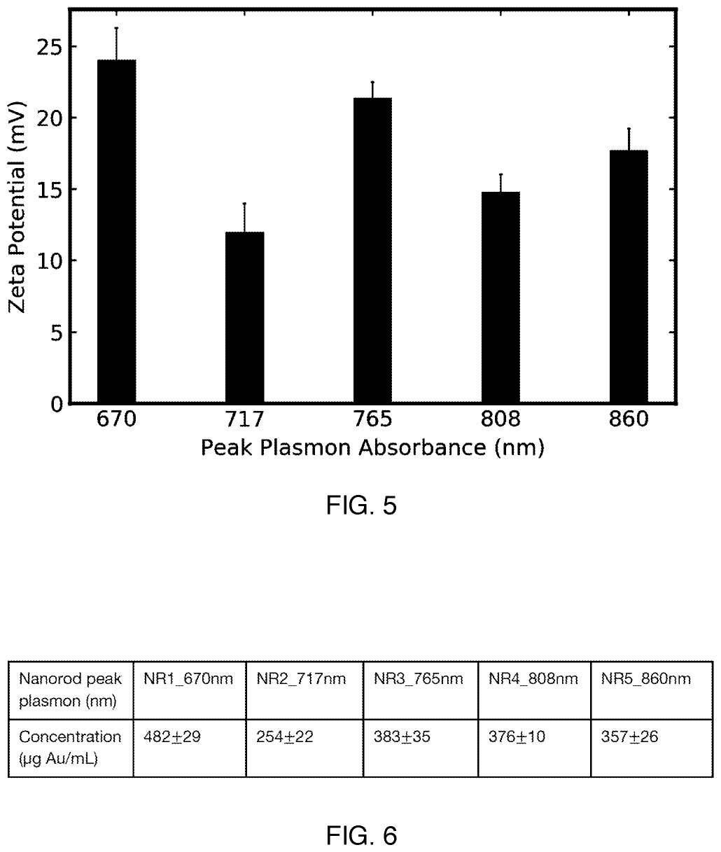

Gold nanorods with longitudinal plasmon resonances ranging from 670-860 nm, governed by their aspect ratio, were colloidally synthesized via a seeded growth: reduction of HAuCl₄ with sodium borohydride creates a spherical seed solution. The seed solution and additional gold salt is mixed with AgNO₃, HCl, ascorbic acid, hexadecyl(trimethyl)ammonium bromide (CTAB), and sodium oleate for nanorod formation. The target aspect ratio is controlled by the concentrations of each agent. All nanorods are coated with surfactants sodium oleate and CTAB, giving them a slightly positive charge, as confirmed by zeta potential measurements (see FIG. 5). UV-vis absorption spectra (FIG. 1D) and transmission and scanning electron micrographs (TEM and SEM) of the gold nanorod samples (FIG. 1E) confirm the red-shifting of the longitudinal plasmon resonance peak with increasing aspect ratio.

We varied the bacteria and Au nanorod mixing ratio and preparation conditions to obtain maximally enhanced SERS signatures while keeping the bacteria viable. Importantly, since the CTAB surfactant from the gold nanorods is cytotoxic, we tested the number of water washing steps needed for aggregation-free nanoparticle dispersion with minimal surfactant coverage to preserve bacterial cell viability. We find that cell viability is maintained with one washing step, as evidenced by cell growth; additional washing steps lead to comparable bacterial viability but significant nanorod aggregation. We also investigated the concentration of nanorods to bacteria, spanning 10%, 30%, 50%, and 100% (double volume of nanorod to bacteria) volumetric ratios. All these ratios maintain cell viability and show significant SERS, with the highest signal generally observed for the largest ratio. Hence, 1:1 volumetric ratio (5 μL of 10⁹ cells/mL bacteria with 5 μL of NR) is used for ease of experimental workflow.

Using a 785 nm laser, we obtained strongly enhanced SERS signatures from the liquid wells with bacteria and nanorods compared to bacteria-only samples. All measurements are obtained using a 5×, 0.12NA objective lens with 30 μm focal spot, experimental conditions chosen to maximize translation to point-of-care clinical applications (i.e., portable, low-cost systems).

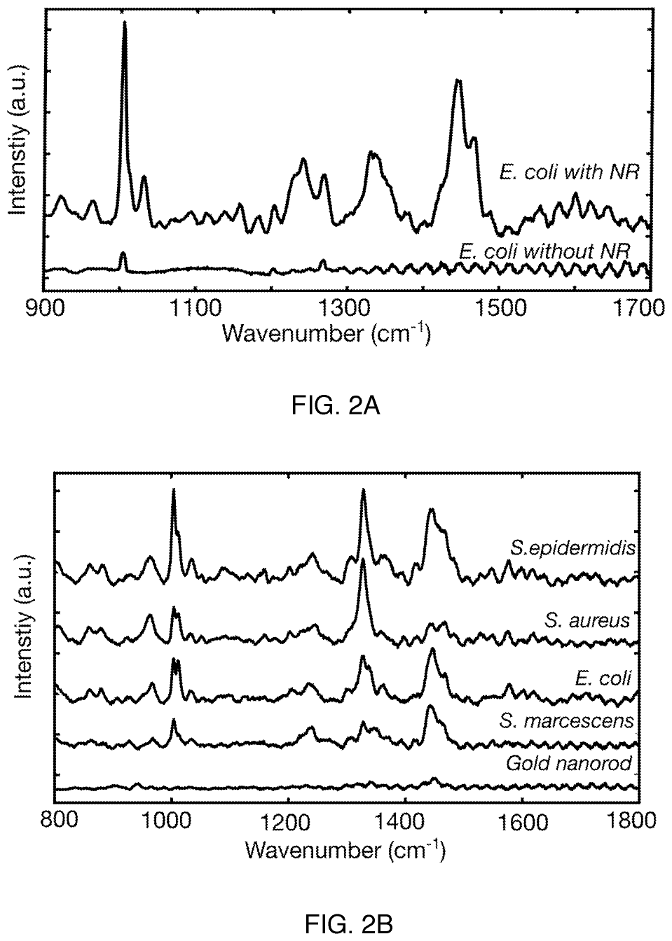

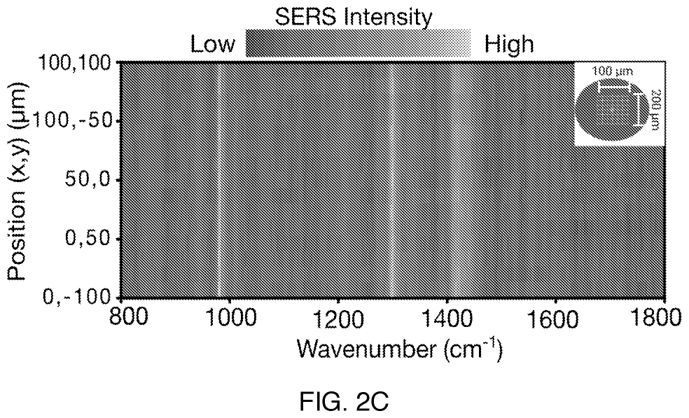

FIGS. 2A-C show spectral enhancement consistency across large surface area of the well and among multiple liquid-SERS chambers. FIG. 2A is a comparison of E. coli Raman spectra with and without the 670 nm nanorods, acquired at 60 s with 5 accumulation, showing no significant spectral signature from liquid E. coli samples without nanorods. FIG. 2B shows liquid-SERS spectra of the four bacterial species mixed with the 670 nm nanorods with the average and standard deviation of 15 measurements at 10 s acquisition across 100×200 μm² dimension on the well (inset in FIG. 2C). FIG. 2C is a heatmap plot of the S. epidermidis spectra shown in FIG. 2B highlighting the consistency of enhancements across the large area. Each row is a spectrum recorded from unique locations on the well. The inset is a schematic showing the xy map region scanned on the liquid well.

FIG. 2A shows representative SERS spectra from E. coli suspended in water (˜10⁹ cells/mL, from log-phase of bacteria culture) showing the significant Raman signal enhancement achieved in liquid samples when mixed with nanorods compared to E. coli only. FIG. 2B shows the average spectra, each collected with 10 s acquisition time, from the four bacterial species E. coli, S. marcescens, S. aureus and S. epidermidis, respectively. With the 670 nm resonant nanorods, bacterial signature spectral peaks near 1000 cm⁻¹, 1350 cm⁻¹, and 1500 cm⁻¹ are observed. The liquid-SERS signatures are nearly identical with the SERS signatures seen with dried specimens; we note that in both dried and liquid cases, the SERS results from a combination of cellular structures (membrane proteins, lipids and intracellular components) as well as excreted metabolites (including metabolites resulting from stress responses to nutrient-poor aqueous media, as in our well). As FIGS. 2A-B show, spectra from the samples without nanorods have no signal under the same interrogation settings; hence detectable spectra are attributed to enhancement from nanorods.

Next, we collected SERS spectra across a 100×200 μm² region within our liquid well. Again, using our 670 nm-resonant nanorods, we obtain uniform Raman signal enhancement for each of the four bacteria, as shown in FIG. 2C. Importantly, the peak signals vary by ˜25% across the entire scanned area, which is reasonable given the size of the area scanned and the large spot size of 30 μm. We also tested the well-to-well reproducibility by using S. epidermidis mixed with our 860 nm-resonant nanorods. Each liquid well shows comparable spectral enhancement with the mean of measurements across the 100×200 μm² area across five separate wells with a variation of ˜15%.

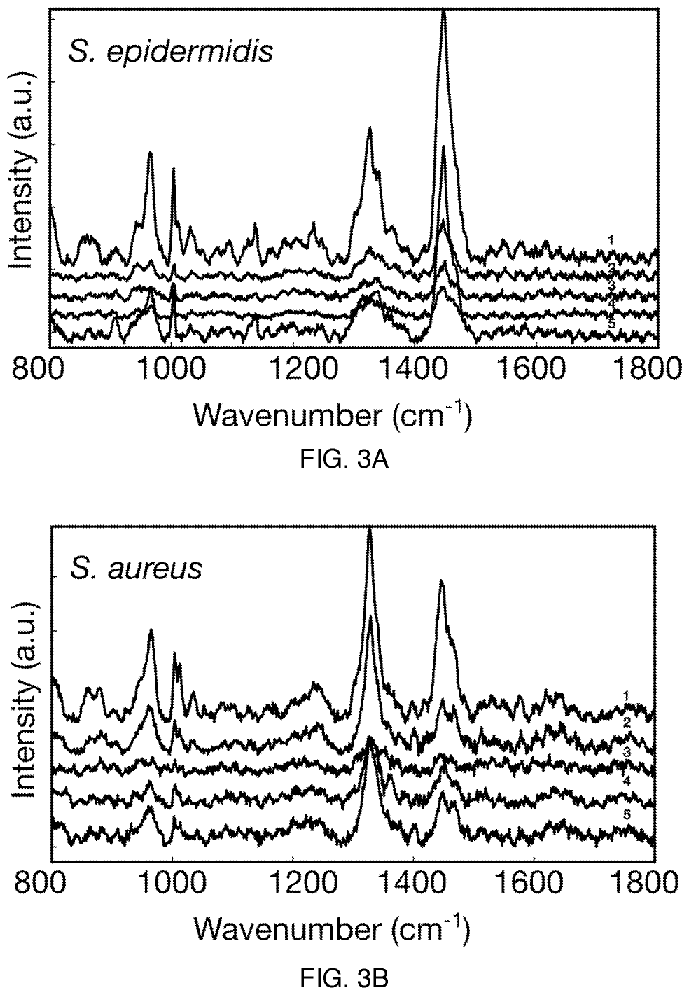

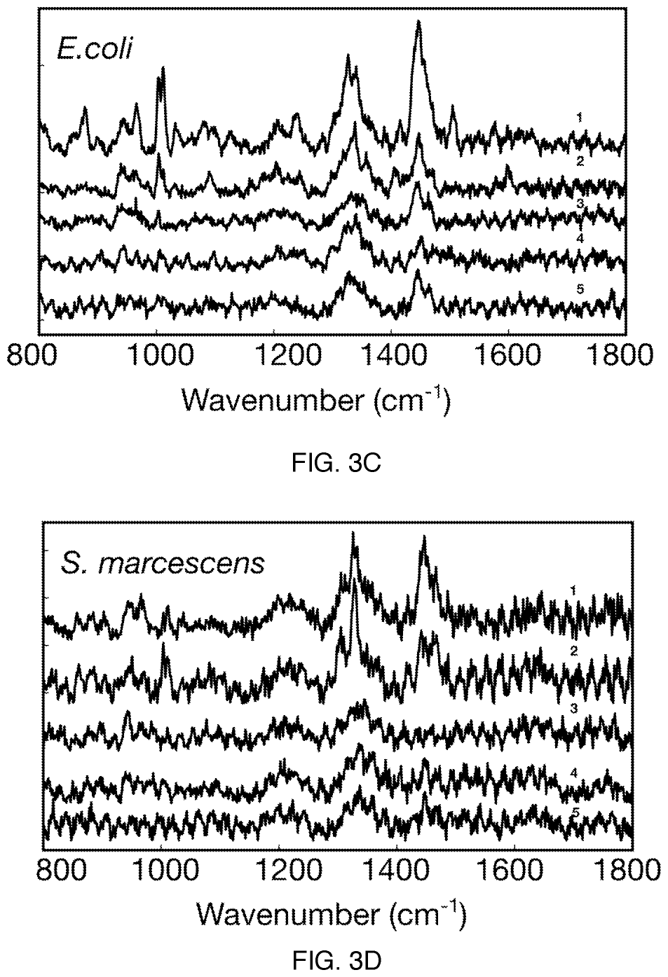

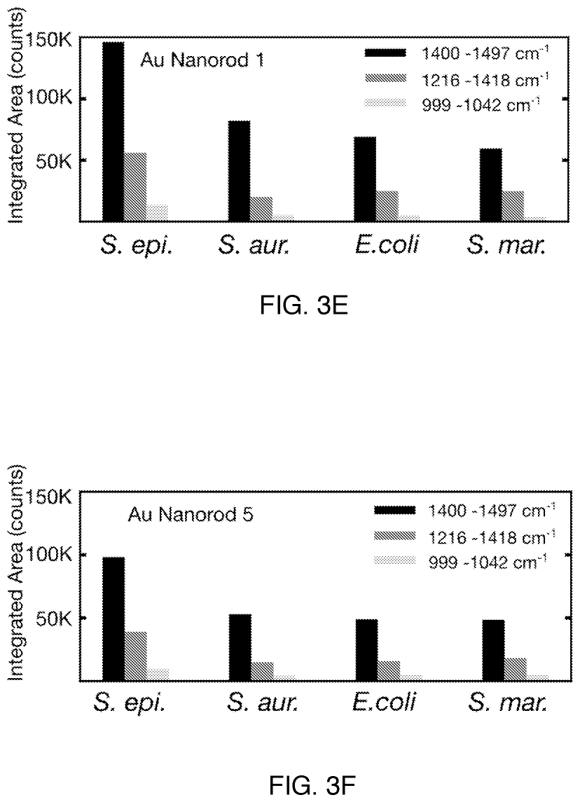

FIGS. 3A-F show SERS spectra comparison across nanorods and bacteria. FIG. 3A shows S. epidermidis spectra combined with the five NRs, numbered with reference to FIG. 1D, and arranged such that most blue shifted is on top and most red shifted at the bottom. FIGS. 3B, 3C, 3D show the same for S. aureus, E. coli, and S. marcescens respectively. FIGS. 3E-F show area under the peak values for signature bacterial peaks near 1000 cm⁻¹, 1300 cm⁻¹, and 1500 cm⁻¹ showing higher values for S. epidermidis and S. aureus which have higher negative surface charge compared to E. coli and S. marcescens. The bar plots shown are for the 670 nm (FIG. 3E) and 863 nm (FIG. 3F) resonant nanorods.

We explored how each nanorod with specific longitudinal plasmon resonance peaks enhances the liquid SERS signals. We considered the five NRs of FIG. 1D and constructed 30 distinct liquid wells, each containing 1:1 bacteria:NR volumetric ratio mixtures. These NR aspect ratios are chosen such that their peak plasmon resonance wavelength spans the relevant optical ranges for SERS including incident illumination (785 nm) and the bacterial Raman shift region (˜835-915 nm). As seen in FIGS. 3A-F, all NR samples show strong SERS signatures. Interestingly, the NR-to-NR variability in signal is comparable to the well-to-well signal variability for a given aspect ratio and bacteria sample. Therefore, within the error of these measurements, all NRs show strong enhancement.

The lack of trend in enhancement factor with NR aspect ratio (peak plasmon resonance) counters standard intuition from dried SERS measurements. In liquids, the nanorods are fairly uniformly distributed throughout the liquid volume. Hence, both incident and scattered light strongly interact with the nanorods as light travels across the liquid volume beyond the measurement plane. This interaction becomes particularly strong when the nanorod resonance overlaps with either the excitation wavelength or the bacteria Raman scattering wavelengths. Accordingly, a “competition between extinction and enhancement” effect as described in the literature arises, compromising the expected higher enhancement at these wavelengths. On the contrary, when the

NR resonance is blue shifted away from these regions of strong competition, the overall setup shows a relatively higher enhancement profile.

Interestingly, higher counts are observed for the NR with the lowest aspect ratio, most blue shifted from the laser excitation. Such enhancements have also been corroborated by other liquid-SERS studies on trap-coated methylene blue molecules and attributed to reduced extinction effects. As discussed above, when using nanorods with resonances matched to the incident laser or the Raman region, both the laser and the Raman scattered light are absorbed by the nanoparticles themselves before reaching the bacterial cells and the detector respectively. However, when using blue shifted nanorods, these extinction effects are avoided. Still, within the variability of multiple measurements from distinct wells, we have not observed a statistically significant difference for SERS from rods of any size. In addition, it can be seen from FIGS. 3A-F that the most redshifted nanorods still contribute to signal enhancement significantly in S. epidermidis and S. aureus cases. Our results indicate that all nanorod aspect ratios provide significant enhancement for recording SERS signatures from liquid bacterial samples compared to bacteria-only samples suspended in water.

Gram-positive and highly negatively charged S. epidermidis and S. aureus overall show more distinct, higher intensity spectra than the Gram-negative E. coli and S. marcescens species. Specifically, S. marcescens shows consistent reduced spectral signal intensity across the nanorods tested and multiple trials while E. coli tends to be lower or comparable in repeat studies. In our study the selected Gram-negative species harbor lower negative surface charge, with E. coli and S. marcescens having negative surface charge densities of ˜0.01 and ˜0.04 (r/e (−)/10⁻⁶ mm²) respectively while the Gram-positive S. epidermidis and S. aureus have negative surface charge densities of ˜1 and ˜0.3 (r/e (−)/10⁻⁶ mm²). S. epidermidis, in particular, has nearly two orders of magnitude higher negative charge density which could significantly contribute to stronger electrostatic interactions with the positively charged nanorods. FIGS. 3E-F illustrate the higher enhancement observed in higher surface charge harboring species. We plot the integrated intensity for spectral peaks near 1000 cm⁻¹, 1300 cm⁻¹ and 1500 cm⁻¹ wavenumbers for two sets of nanorods. Across all nanorods, significantly higher SERS signal is observed for S. epidermidis and S. aureus which have higher negative surface charge compared to E. coli and S. marcescens.

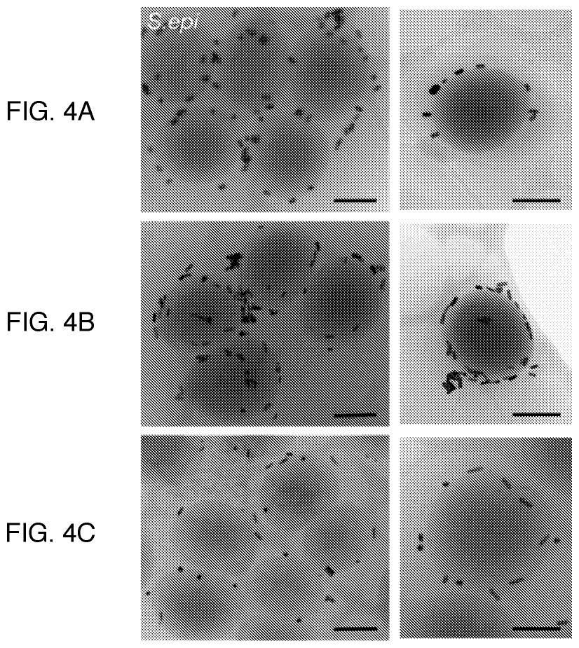

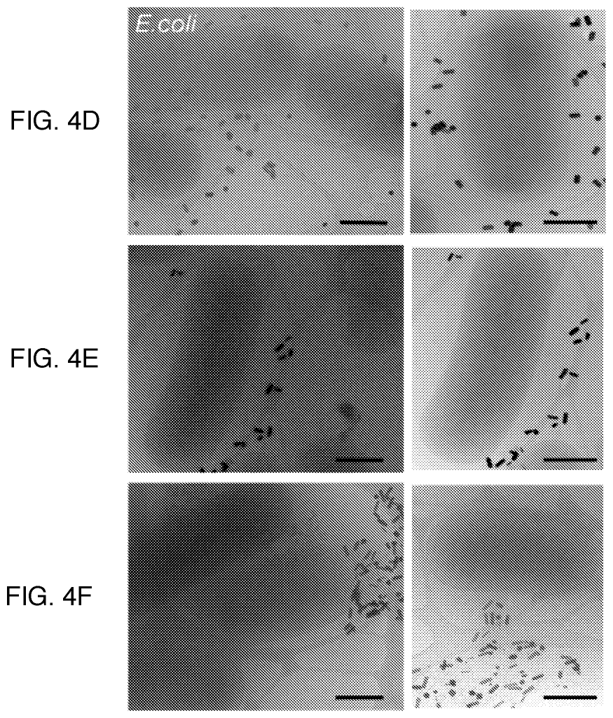

FIGS. 4A-F show interaction between bacterial cells and nanoparticles and its effect on Raman signal enhancement. FIGS. 4A, 4B, 4C are Cryo-EM images of S. epidermidis mixed with NRs with longitudinal plasmon resonance peaks: 670 nm, 717 nm, 860 nm, respectively. Similarly, FIGS. 4D, 4E, 4F are Cryo-EM images of E. coli mixed with NRs with longitudinal plasmon resonance peaks: 670 nm, 717 nm, and 860 nm, respectively. The nanoparticles show tight binding to S. epidermidis cell surface compared to E. coli cell surfaces as the former has higher negative surface charge. Scale bar indicates 500 nm.

To better understand the local interaction between the cells and NRs in our liquid well, we performed cryo-EM. We froze our liquid bacteria-NR mixture onto a 200-mesh Cu lacey carbon grid to image the nanorod distribution on the bacteria as-is in liquid. We imaged with a low dose at −177° C. The cryo-EM images show distinct differences in the arrangement of NRs around Gram-negative and Gram-positive bacteria. As seen in FIGS. 4A-C, the NRs surround Gram-positive S. epidermidis by closely binding to its membrane surface, whereas in the case of Gram-negative E. coli (FIGS. 4D-F), the nanorods do not show specific adherence to the bacterial membrane surface. The observed close binding is because of the positive surface charge of the NRs (resulting from the CTAB surfactant on their surface, confirmed by the zeta-potential measurements shown on FIG. 5) interacting with the negative surface charge on the bacterial membrane. In particular, S. epidermidis has ˜30× higher negative charge with significantly smaller surface area compared to E. coli. The large charge distributed over a small surface area allows S. epidermidis to have nearly two orders of magnitude higher charge density than E. coli, resulting in a stronger electrostatic interaction with the positively charged NRs.

We also confirmed no significant change in the full-width half max and plasmon peak location (there is a slight red shift with addition of bacteria which can be attributed to the slight increase in refractive index of the solution) of the nanorods when mixed with the bacteria species. Interestingly, the spectral data from three isolated trials comparing the SERS enhancement for S. epidermidis and E. coli do not show statistically significant differences upon repeated measurements. Therefore, although differences in surface charge lead to different NR coverage on individual bacterial cells and thus variations in enhancement, the NRs still effectively enhance the overall SERS signal as the NRs and cells can freely interact in the dynamic liquid environment. Furthermore, bacterial NR mixtures were measured within a few minutes, after 5 hrs and after 24 hrs, were tested and show new peaks arising near 1600 cm⁻¹ with time (24 hrs) which can be attributed to cellular death showing the potential use of such liquid wells for monitoring live cell changes across time.

In summary, we have demonstrated a simple and robust liquid Raman well setup that enables uniform, large-area SERS enhancement of bacteria in liquid, while maintaining cell viability and prevents exposure to pathogenic bacteria under investigation. Our results show that bacteria with higher negative surface charge better attract the positively charged Au NRs, leading to higher SERS enhancements. Furthermore, several NRs with longitudinal plasmon resonance peaks with peaks ranging from 670-870 nm can be used to obtain SERS enhancements despite the differences in peak location.

Our work could facilitate clinical translation of Raman-based diagnostics. For example, in the realm of antibiotic testing, combination therapy is gaining traction as a more effective treatment approach compared to single antibiotic treatment; a reliable liquid-SERS environment integrated with inflow and outflow channels could be used to test the effect of combined antibiotic therapy on pathogens by monitoring dynamic changes in the bacteria directly from Raman spectral changes. Liquid-SERS could also enable real-time drug susceptibility testing on cancer cells, without the need for fixing or freezing of the cells, potentially at the single-cell level. Additionally, liquid SERS could enable viable bacterial identification at low concentrations—especially important for bacteria that are difficult to culture, such as in microbiome studies and environmental microbiology. Lastly, in pharmaceutical applications such as drug discovery, liquid-SERS could be used to monitor chemical composition of excreted factors in combined cultures while maintaining cellular viability. Our simple sample preparation platform and rigorous understanding of liquid bacterial SERS hopefully provides a foundation for such studies and more, en-route to low-cost, point-of-care diagnostics.

### C) Supplemental Information

### C1) Materials and Methods

### Gold Nanorod Synthesis and Characterization:

Hexadecyl(trimethyl)ammonium bromide (CTAB) and sodium oleate (NAOL) coated gold nanorods were synthesized following previously described protocols. The nanorods were cleaned by centrifuging 1 mL aliquots twice at (9000 rpm, 20 min) and were concentrated down to 100 μL volumes. Absorption spectra were recorded using a Cary 5000 UV-vis-NIR spectrometer. Scanning electron microscopy images were taken using FEI Magellan 400 XHR Scanning Electron Microscopy (SEM). Transmission electron microscopy images were taken using FEI Tecnai G2 F20 X-TWIN Transmission Electron Microscope (TEM) microscope.

Bacteria Culturing and Preparation: E. coli ATCC 25922, S. marcescens ATCC 13880, S. aureus ATCC 29213, and S. epidermidis ATCC 12228 were grown from frozen stocks on to Trypticase Soy Agar 5% Sheep Blood 221239 BD plates. A single colony was seeded in 10 mL Lysogeny broth (LB) culture medium and incubated at 37° C. shaking at 300 rpm for 15 hrs using Thermo Scientific MaxQ 4450 incubator. 1 mL of culture (˜10⁹ cells/mL from log phase) was washed with water three times at 6000 rpm for 3 min using a mySPIN™ 6 Mini Centrifuge. 5 μL of the bacterial sample was mixed with 5 μL of NRs to a total volume of 10 μL (maximum volume accommodated by the well given the hole size used).

Liquid SERS well fabrication: The well has a silicon wafer base made, a ˜100 μm tall double-sided adhesive layer created using 4 layers of 3M™ double-sided tape and a glass coverslip. Double-sided adhesive is punched using the Bostitch EZ Squeeze™ 1-Hole Punch single-hole puncher. The adhesive is cut to a square shape and pasted on the silicon wafer. 10 μL of mixed 5 μL bacteria and 5 μL NR sample is filled in the hole and completely sealed with a glass coverslip. The well is placed on a support glass slide for Raman interrogation.

Raman spectroscopy: Raman spectra was collected using the Renishaw inVia™ confocal Raman microscope. The excitation wavelength for all measurements was 785 nm with a 30 s acquisition time unless specified otherwise. The Raman shift from 750 cm⁻¹ to 1800 cm⁻¹ was collected using 1200 gr/mm grating. Laser light was directed to and Raman scattered light was collected from the sample using a 5×, 0.12NA objective with spot size of 30 μm. Laser power at the sample was 30 mW. Bacterial NR mixtures were measured within ˜1 hr of sample preparation.

Spectral Data Processing: Ipython (Jupyter Notebook) was used to process spectral data. Background was subtracted using a polynomial fit with degree 70. The specific package used and code line is: peakutils.baseline(y, deg=70, max_it=1000, to1=0.0001). Note the need for a higher degree polynomial rises from a typical instrumental background that is difficult to fit with lower degree fits. Area under the curve is computed by using the trapezoid rule and dx=1.3.

Cryo-EM: For cryo-EM imaging, 3 μL of a freshly-prepared solution of Au with bacteria (diluted 2×) was applied onto a 200-mesh Cu lacey carbon grid (LC200-Cu, Electron Microscopy Sciences, Hatfield, PA), which was first glow-discharged for 25 seconds in a PELCO easiGlow (Ted Pella, Inc.). The grid was then blotted with a filter paper for 1 second, followed by plunge-freezing into liquid ethane using a Leica EM GP automatic plunge freezer (Leica Microsystems). This grid was transferred under liquid nitrogen to a Gatan cryo-holder using a cryo-transfer station to keep the sample under −170° C. and to minimize contamination. The sample was imaged at a temperature of −177° C. using low-dose imaging and a Gatan K2 Summit direct electron detector (Gatan, Inc.) on a Thermo Fisher Scientific Tecnai TF20 electron microscope operated at 200 kV.

Zeta potential measurements: 15 μL of purified gold nanorod solutions were dispersed in 1 mL of HEPES (4-(2-hydroxyethyl)-1-piperazineethanesulfonic acid) buffer. The measurements were taken using Malvern Dynamic Light Scattering (DLS, Westborough, MA) system. Three measurements per sample were collected with the average and standard deviation plotted in FIG. 5. All measurements were performed at 25° C.

Gold NR concentration measurements: Inductively coupled plasma mass spectroscopy (ICP-MS, Thermo Scientific XSERIES 2) was used to measure the concentration of gold in the NR solutions. 5 μL of the synthesized and purified nanoparticles were dissolved in 100 μL of highly concentrated hydrochloric acid (HCl, 12.85 M). Samples were diluted for ICP measurements to 5 mL using Millipore water. Measurements were repeated three times and the average concentration was used for each sample as shown in FIG. 6.

### C2) Gold Nanorods for SERS Application

The principle behind the Raman signal enhancement in SERS is the local confinement of electric field from both the incident light and the Raman scattered light by the SERS substrate, which in total results in fourth order enhancement in the local electric field (E⁴). Colloidal nanoparticles in general are the most convenient forms of metallic SERS substrates because of the relative ease and scalability of synthesis. Particularly, nanoparticles with sharp tips, such as gold nanorods, provide large Raman spectral enhancement factors, which make them ideal for application for liquid biological specimen. Gold nanorods (NR) have been well-characterized in the past, allowing for reproducible and controlled synthesis. The NR aspect ratio (length:width) can be varied to control scattering and absorption properties to obtain desired Raman signal enhancement. The enhancement could range from providing distinct Raman spectra to inducing targeted cell damage via local heating. In addition, advances in SERS and Infrared spectroscopy substrates such as metal liquid-like plasmonic arrays, graphene based nanodot arrays, and core-shell alloy substrates promise versatility and significant improvement in enhancement performance in liquid. Although direct comparisons with previous reports on dried bacterial NR mixtures are difficult to make, our results overall show comparable intensity counts. For instance, antibody tagged NRs were reported to have counts as high as 18,000 a.0 for ˜10⁵ cells/mL using 100 mW, 60 s acquisition time and a 20× objective lens (compared to ˜20,000 a.u for ˜10⁹ cells/mL using 30 mW, 30 s acquisition time and a 5× objective in our measurements). Similarly, another recent work using 50 mW reported signal intensities as high as 10,000 a.u. for ˜10⁸ cells/mL compared to no spectral signature without mixing nanorods.

### C3) Zeta Potential

FIG. 5 shows zeta potential measurements. The NRs are positively charged with zeta potential values averaging close to 20 mV. The error bars show the standard deviation between three measurements per sample.

### C4) Concentrations of NRs Tested

FIG. 6 shows the NR concentrations used in this work. These concentration variations have been shown not to impact the overall liquid-SERS enhancements. The higher reported concentrations for NR1 can be attributed to the packing density of the smaller sized gold particles in the volume of sample tested compared to larger sized particles.

1. A method of monitoring one or more biological target species in a liquid, the method comprising:
disposing nanoparticles in the liquid, wherein the nanoparticles are treated with a surfactant configured to provide an electrostatic attraction between the nanoparticles and at least a selected species of the one or more biological target species;
illuminating the liquid with Raman pump light;
observing Raman-scattered light from the liquid, wherein the Raman-scattered light is scattered by the selected species in an interaction that is surface-enhanced by the nanoparticles.

2. The method of claim 1, wherein the selected species has a negative surface charge and wherein the surfactant is configured to provide a positive surface charge.

3. The method of claim 1, wherein the selected species has a positive surface charge and wherein the surfactant is configured to provide a negative surface charge.

4. The method of claim 1, wherein the electrostatic attraction between the nanoparticles and the selected species is label-free.

5. The method of claim 1, wherein the observing Raman-scattered light from the liquid is performed continuously in real time for a viewing duration.

6. The method of claim 1, wherein the liquid is selected from the group consisting of: plasma, serum, whole blood, saliva and sputum.

7. The method of claim 1, wherein the Raman scattered light is from a single living cell of the selected species.

8. The method of claim 1, wherein the selected species is a bacterium species.

9. The method of claim 8, further comprising performing an assay of one or more antibiotics vs. the bacterium species using the Raman-scattered light.

10. The method of claim 1, wherein the selected species is a cancer cell species.

11. The method of claim 10, further comprising performing an assay of one or more anti-cancer agents vs. the cancer cell species using the Raman-scattered light.

12. The method of claim 1, wherein the selected species is a mammalian cell species.

13. The method of claim 13, further comprising performing an assay of one or more drugs vs. the mammalian cell species using the Raman-scattered light.

14. The method of claim 1, wherein the one or more biological target species includes one or more species selected from the group consisting of: mammalian cells, cancer cells, bacteria, viruses, nucleic acids and proteins.
